(19) United States
(12) Patent Application Publication
(10) Pub. No.: US 2006/0183222 A1
(43) Pub. Date: Aug. 17, 2006

(54) Method for culturing neurons, neuron culture substrate, neurons, neuron system, and method for manufacturing neuron system

(76) Inventors: Kosuke Kuwabara, Hitachi (JP); Akihiro Miyauchi, Hitachi (JP); Masatsugu Shimomura, Sapporo (JP); Masaru Tanaka, Sapporo (JP); Hiroshi Yabu, Sapporo (JP); Akinori Tsuruma, Sapporo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/354,874

(22) Filed: Feb. 16, 2006

(30) Foreign Application Priority Data
Feb. 17, 2005 (JP) 2005-041381

(51) Int. Cl. C12N 5/08; C12M 3/00

(52) U.S. Cl. 435/368; 435/289.1

(57) ABSTRACT

There is provided a method for controlling morphological growth of neurons by specifically controlling the surface configuration of a substrate. There is also provided a neuron culture substrate necessary for application of the method and neurons controlled in morphological growth. The method for culturing neurons include, providing a culture medium and neurons on a cell culture substrate 1 and culturing the neurons in corresponding culture conditions, wherein the culture surface of the cell culture substrate 1 has a plurality of protrusions 4, and the shape, interval or both is controlled to control morphological growth of the neurons.

### BACKGROUND OF THE INVENTION

The present invention relates to a method for culturing neurons, a neuron culture substrate for use in the method, and neurons cultured by the method. More particularly, the present invention relates to a method for culturing neurons while controlling morphological growth of the neurons, a neuron culture substrate for use in the method, and neurons cultured by the method.

Recently, a cell culture technology has been frequently used in the medical field including regenerative medicine and medical transplantation fields. More specifically, at present, the cell culture technology is applied to skin transplantation. Furthermore, with advance of the technology, the cell culture technology has been increasingly applied to the cornea, teeth, bone, and further to more complicated organs such as tissues.

In particular, studies on neurons have been extensively conducted with the view to recuperating the function of defective neurons and reforming the neural circuit, by taking advantage of self-organization ability intrinsic to the neuron. Such regeneration of nerve circuits is considered effective in treating Parkinson's disease and Alzheimer's disease.

The neurons extend axons from each other to construct a network, thereby carrying out various functions.

However, conventional culture vessels such as Petri dishes formed of glass and a resin for culturing neurons are not designed for growing neurons while controlling morphology. Therefore, neurons cannot be cultured while controlling morphological growth in such a conventional culture vessel. Cells can be cultured while controlling morphological growth by adding an inducing substance and a suppressing substance such as cytokines to the medium; however, such substances may bring side effects upon the cells. For this reason, it may not be permissible to say that such a method is favorable, depending upon application of the cells.

Accordingly, it has been increasingly required to develop a method for culturing neurons while controlling morphological growth without particularly adding an inducing substance or suppressing substance.

Under the requirement, for example, Japanese Patent No. 3038365 (paragraph 0022-0027) proposes a method for culturing neurons while controlling the contour of neurons by using a substrate, which is made of silicone (preventing cells from adhering onto the culture substrate) and patterned by optical lithography. High-resolution patterning can be performed by use of a modification method based on optical lithography. According to the disclosure of the patent, the morphology of a neuron network can be controlled by culturing the neurons only on the region of a culture substrate where a non-adhesive material to cells is not applied.

Furthermore, for example, National Publication of International Patent Application No. 10-500031 (pages 6 to 12) proposes a method for controlling the extension direction of axons protruding from neurons by culturing the neurons on a culture substrate having grooves arranged at minute intervals in the surface. According to the method disclosed in this publication, grooves are formed in the surface of a culture substrate by use of a mold previously prepared, so that the number of manufacturing steps can be reduced.

### SUMMARY OF THE INVENTION

However, the method disclosed in the Japanese Patent No. 3038365 has a problem. Since a culture substrate is manufactured by optical lithography, the number of manufacturing steps increases, raising manufacturing cost.

Also, the method disclosed in National Publication of International Patent Application No. 10-500031 has a problem. The grooves are effective in controlling morphological growth only in the axonal extension direction and not effective in other directions. Therefore, the method is insufficient to control growth of cells to form an appropriate network. Furthermore, to prevent adhesion and extension of cells outside a desired region (groove region), the surface of the region outside the groove region must be treated by a different method from that applied to the groove region. After all, the number of manufacturing steps increases, raising manufacturing cost.

The aforementioned two methods are effective in controlling the shape of axons of neurons; however, not directly conducive to controlling the shape of the cell body.

The present invention has been achieved in view of the aforementioned problems and directed to providing a method of controlling the morphological growth of neurons without particularly adding an inducing substance and a suppressing substance. The present invention is further intended to provide a neuron culture substrate and neurons controlled in morphological growth.

To solve the aforementioned problems, there is provided a method for culturing neurons comprising providing a culture medium and neurons on a neuron culture substrate made of an organic polymer and culturing the neurons in corresponding culture conditions, wherein a culture surface of the neuron culture substrate has a plurality of protrusions, and a shape, an interval or both of the plurality of protrusions are controlled to control morphological growth of the neurons.

More specifically, the morphological growth of the neurons varies depending upon the relationships between the shape and interval of the protrusions, as mentioned below.

First, when the equivalent diameter of protrusions and interval of the protrusions are smaller than the diameter of the neurons to be cultured and the diameter of the neurites extending from the neurons, the adhesion strength between the neurons and the protrusions can be increased. Therefore, compared to the morphological growth of the neurons cultured on a general flat substrate, the resultant neurons have flat cell bodies and neurites increased in number and thickness.

Second, in the case where the equivalent diameter of the protrusions and the interval of the protrusions on a neuron culture substrate are smaller than the diameter of the neurons to be cultured and the interval of the protrusions is larger than the diameter of the neurites extending from the neurons, the protrusions facilitate the straightforward extension of the neurites extending from the neuron body, allowing the neurites to grow longer. Therefore, compared to the morphological growth of the neurons cultured on a general flat substrate, the neurites tend to extend straightforward and longer.

Third, in the case where the equivalent diameter of the protrusions is smaller than the diameter of neurons and interval of the protrusions falls within the range of 0.4 to 2 times, more preferably, 0.6 to 2 times as large as the diameter of the neurons to be cultured, the neurites are prevented from extending from the neuron body, with the result that the neurons shrink. Therefore, compared to the morphological growth of neurons cultured on a general flat surface, the resultant neurons have a small diameter and extremely short neurites.

According to the present invention, the morphological growth of neurons can be controlled without particularly adding an inducing substance or a suppressing substance. Since the morphological growth can be controlled by specifying the shape of the protrusions and interval of them. Therefore, it is not necessary to particularly apply a surface treatment to a surface of a neuron culture substrate. Hence, the neuron culture substrate for controlling morphological growth of neurons can be manufactured simply and inexpensively.

Other objects, features and advantages of the invention will become apparent from the following description of the embodiments of the invention taken in conjunction with the accompanying drawings.

### BRIEF DESCRIPTION OF THE DRAWINGS

FIG. 1 is a longitudinal sectional view for explaining a method for culturing neurons according to the present invention;

FIG. 2 is a perspective view of a neuron culture substrate according to an embodiment of the present invention;

FIG. 3 is a partially enlarged perspective view of Region A of the neuron culture substrate shown in FIG. 2;

FIG. 4 shows sectional views of a neuron culture substrate for explaining the steps of a method for manufacturing it by nano-imprinting;

FIG. 5 is a view for explaining a method for culturing a neuron according to Embodiment 1;

FIG. 6 is a view for explaining a method for culturing a neuron according to Embodiment 2;

FIG. 7 is a view for explaining a method for culturing a neuron according to Embodiment 3;

FIG. 8 shows a top view of a neuron culture substrate;

FIG. 9 shows a top view of another neuron culture substrate;

FIG. 10 shows a top view of still another neuron culture substrate; and

FIG. 11 is a top view of a neuron culture substrate manufactured in Example 1.

### DESCRIPTION OF PREFERRED EMBODIMENTS

Best modes (hereinafter referred to as “embodiments”) for carrying out the invention will be explained, if necessary, with reference to the accompanying drawings. In the following description, like reference numerals are used to designate like structural elements and any further explanation is omitted for brevity's sake. Note that in the drawings, like reference numerals are used to designate like structural elements; however, shapes and sizes of like structural elements are not always identical.

<Method for Culturing Neurons>

The present invention has been achieved based on the finding that neurons can be cultured while controlling the morphological growth by placing a culture medium and the neurons on a neuron culture substrate having a plurality of protrusions having a predetermined shape and arranged at predetermined intervals on the culture surface of the substrate, and culturing under corresponding culturing conditions.

The term “morphological growth of neurons” means various morphological characteristics of neurons grown on a neuron culture substrate. Examples of the morphological characteristics of neurons grown include, but not limited to, adhesion between neurons and the neuron culture substrate, size of cell bodies, shape of cells such as flat, spherical, and spindle shapes, the thickness and length of neurites, branching pattern (the number of branches, which position a spine is branched) of neurites, extension direction of neurites, acceleration and suppression (termination) of neuron growth.

In the embodiment, the term “neurites” includes dendrites and axons.

The neurons used in the embodiment are not particularly limited as long as they can be cultured on the surface of a substrate and may be appropriately selected from conventional neurons. For example, when they are applied to a human in the medical field, use may be made of neurons derived from a human, more specifically, neurons derived from the tissue to be transplanted to an affected portion of a recipient. On the other hand, when they are applied to a usage in which a human is not involved, for example, constructing a neuron system such as a neuro device, the neurons may not be derived from a human and may be selected from the neurons derived from various animal species and tissues. The neurons may be isolated from a biological tissue or derived from stem cells by induction differentiation. Alternatively, such neurons may be subcultured and put into use. The neurons may or may not have a proliferation potential and may be derived from a fetus or an adult. In other words, neurons that are used in the embodiment can be appropriately selected from available neurons suitable for the purpose.

In the embodiment, the neurons can be cultured under culture conditions suitable for the neurons appropriately selected from known culture conditions. One skilled in the art would easily select culture conditions suitable for the selected neurons and perform culturing in the selected culture conditions.

Now, general culture conditions for neurons will be explained.

As a medium, use may be made of a known medium containing suitable components for culturing neurons. For example, a commercially available medium for culturing neurons may be used. In this case, serum such as fetal bovine serum (about 10%) may be added to the medium for facilitating fixation of neurons onto a neuron culture substrate. Separately, an inducing substance and a suppressing substance may be added.

However, in the embodiment, to clearly describe an effect of controlling morphological growth of neurons by a method of culturing neurons according to the embodiment, explanation will be made on the premise that culturing is performed in the medium containing no inducing/suppressing substance for controlling morphological growth except for a fixation substance.

As an incubator for culturing cells, use can be made of a CO₂ incubator as used for culturing general cells. In the CO₂ incubator, a CO₂ concentration is set at 5%, a temperature 37° C., and a relative humidity 80%.

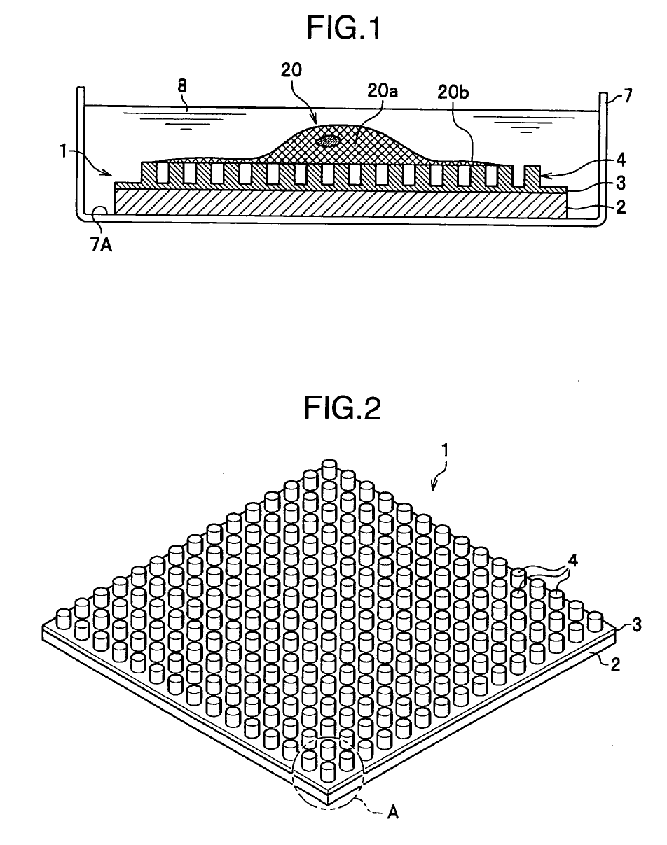

Now, a procedure for culturing neurons will be explained with reference to FIG. 1.

First, neurons 20 are seeded together with a medium 8 on a neuron culture substrate 1.

Then, the neuron culture substrate 1 on which the medium 8 and the neurons 20 are seeded is allowed to stand still in a CO₂ incubator for a predetermined period.

In this step, the neurons 20, which are fixed on the neuron culture substrate 1, are incubated. After fixation of cells, the medium 8 may be replaced with a fresh medium at predetermined time-intervals. Examples of the medium 8 may include a serum medium and non-serum medium and a medium with a supplement and a cytokine added thereto. When a non-serum medium is used, the medium is preferably replaced with a fresh medium at the intervals of 1 or 2 days After culturing for the predetermined period, the neurons 20 are subjected to observation. The predetermined period is not particularly limited and may be varied (extended or reduced) depending upon the desired morphological growth of neurons 20. In this embodiment, the morphology growth of the neurons 20 was observed 7 days after the seeding.

<Neuron Culture Substrate>

In this embodiment, the neurons 20 are cultured while controlling the morphological growth. The culturing is performed on a neuron culture substrate 1 having a plurality of protrusions 4 on the culture surface, as shown in FIG. 2.

As a typical example of such a substrate having protrusions 4 applicable to the embodiment, mention may be made of a functional substrate described in JP-A-2004-170935, which is previously filed by the applicant of the present invention. The functional substrate is composed of a first substrate made of an organic polymer and minute columnar projections (made of the organic polymer) protruding from the base body. The minute columnar projections have an equivalent diameter of 10 nm to 500 μm and a height of 50 nm to 5,000 μm, and characterized in that the ratio (H/D) of the height (H) to the equivalent diameter (D) is not less than 4.

In the embodiment, use is made of a culture substrate having a plurality of protrusions on the culture surface, and further the shape and interval of the protrusions are specified in order to control the growth of neurons 20 in accordance with a desired morphological growth.

FIG. 2 is a perspective view of a neuron culture substrate according to this embodiment. As shown in FIG. 2, the neuron culture substrate 1 is composed of a substrate base 2, a resin layer 3 formed on the upper surface of the substrate base 2 and a plurality of protrusions 4 integrally formed on the resin layer 3.

As shown in FIG. 1, the neuron culture substrate 1 is placed on the bottom surface 7A of a culture vessel 7. When a medium 8 is dispensed to the culture vessel 7, the culture surface of the neuron culture substrate 1 is soaked in the medium 8. In this case, the neuron culture substrate 1 may be detachably or permanently placed on the bottom surface 7A. The neuron culture substrate 1 is not limited to a flat board. More specifically, use may be made of a flexible sheet-form substrate, a curved substrate and a three-dimensional substrate such as spherical and columnar substrate.

Furthermore, the neuron culture substrate 1 may have a concave portion to receive the medium 8. The neuron culture substrate 1, if it is formed like a container such as a Petri dish or a flask, can easily carry the medium 8 supplied. In this case, the culture container 7 is no longer required for culturing.

The shape of the neuron culture substrate 1 may be appropriately selected depending upon the usage of neurons 20 after culture.

<Substrate Base 2>

In the embodiment, the substrate base 2 of the neuron culture substrate 1 may be used as a base for a general neuron culture substrate and may not be particularly limited as long as it can be formed of a material having an appropriate strength. Furthermore, the substrate base 2 may possibly come into direct or non-direct contact with the neurons 20 and the medium 8. In consideration of the possibility, the substrate base is preferably formed of a less cytotoxic and high biocompatible material.

Examples of the material for substrate base 2 include thermoplastic resins such as polyethylene, polypropylene, polyvinyl alcohol, polyvinylidene chloride, polyethylene terephthalate, polyvinyl chloride, polyurethane, polystyrene, ABS resin, AS resin, acrylic resin, polyamide, polyacetal, polybutylene terephthalate, glass tempering polyethylene terephthalate, polycarbonate, modified polyphenylene ether, polyphenylene sulfide, polyether ether ketone, liquid crystal polymer, fluoro-resin, polyarate, polysulfone, polyether sulfone, polyamide imide, polyether imido, and thermoplastic polyimide; and thermosetting resins such as phenol resin, melamine resin, urea resin, epoxy resin, unsaturated polyester resin, alkyd resin, silicone resin, diallyl phthalate resin, polyamide bismaleimide, and polybisamidetriazole. They may be used in the form of a combination of two types or more.

Other than resin compositions mentioned above, use may be made of an inorganic substance including a ceramic such as quartz, glass, alumina, zirconium, or titanium for forming the substrate base.

Furthermore, when a method for culturing neurons according to the present invention is applied to the medical field, the substrate base 2 may preferably be formed of a synthetic product such as an aliphatic polyester (e.g., polylactic acid and polycaprolactone), polyacid anhydride, or synthetic polypeptide; a biodegradable resin including a natural resin such as chitosan and cellulose, or a mixture of not less than these two types of resins. With this constitution, the neurons 20 thus cultured can be transferred to a living body, separately from or together with the neuron culture substrate 1.

<Resin Layer 3>

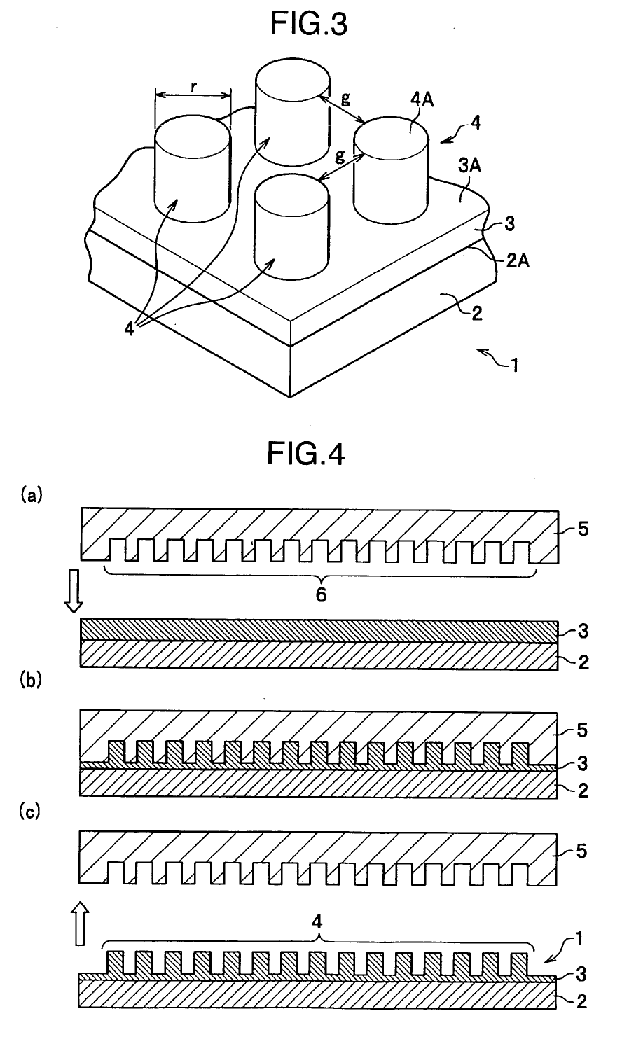

FIG. 3 is an enlarged perspective view of the neuron culture substrate 1 shown in FIG. 2.

In the embodiment, the resin layer 3-is arranged on the upper surface 2A of the substrate base. The material for the resin layer 3 may be appropriately selected depending upon a desired accuracy in processing, surface characteristics, optical characteristics, and strength, etc., and may not be particularly limited. More specifically, the material of the resin layer 3 may be appropriately selected from the resins, resin compositions, inorganic substances and biodegradable resins exemplified as the materials for the substrate base 2.

<Protrusions 4>

As shown in FIG. 3, according to the embodiment, the protrusions 4 on the neuron culture substrate 1 formed on the upper surface 3A of the resin layer 3 are integrally formed with the resin layer 3 and constitute the control regions described later (for example, Embodiments 1 to 3).

The protrusions 4 have a predetermined equivalent diameter r, which is measured on the top surface 4A, and arranged at predetermined intervals g.

The protrusions 4 may be arranged in any manner as long as they are arranged at the predetermined intervals g. The protrusions 4 are arranged preferably in a two-dimensional form such as a tetragonal lattice and a cross-woven lattice in order to produce a uniform effect in the same control region.

The shape of the top surface 4A of the protrusion 4 is not necessarily circular. For this reason, in this embodiment, the size of the top surface 4A is specified by the term “equivalent diameter” instead of the term “diameter”, which implies that the shape is round.

The term “equivalent diameter” means the diameter or length equivalent to the diameter of the top surface 4A of the protrusion 4. When the top surface 4A is a circle, the diameter of the circle is used. In contrast, when it is a square, a length of a side of the square is used. When the shape of the top surface 4A does not satisfy both of them, the diameter of an equivalent circle may be used. The diameter of an equivalent circle is used on the assumption that the shape of the top surface 4A is a circle even though the shape of the top surface 4A is not a circle in a strict sense. For example, an area-equivalent circle diameter is employed on the assumption that the top surface 4A is regarded as a circle having the same area as that of the surface 4A. A circumference-equivalent circle diameter is employed on the assumption that the top surface 4A is regarded as a circle having the same circumference as that of the surface 4A. A circumscribing-circle equivalent circle diameter is employed on the assumption that the top surface 4A is regarded as a circle circumscribing the top surface A. An inscribing-circle equivalent diameter is employed on the assumption that the top surface 4A is regarded a circle inscribing the top surface A. In this manner, a type of equivalent circle can be appropriately selected depending upon the shape of the top surface A.

The equivalent diameter r of a protrusion 4 is preferably smaller than that of a neuron in order to reduce the area in contact with the neuron. With this constitution, when a neuron 20 is placed on the top surface 4A of the protrusion 4, the area of the bottom surface of the neuron in contact with the medium 8 increases, accelerating exchange of a nutritional substance and a waste product between the neuron and the medium 8. In this manner, a predetermined effect can be exerted on controlling of morphological growth of the neuron 20.

In the embodiment, the interval g between protrusions 4 is defined as the shortest distance from a portion of the circumference of the top surface of a protrusion 4 to that of an adjacent protrusion 4.

To explain more specifically, when protrusions are arranged in the form of a two-dimensional tetragonal lattice, as shown in FIGS. 2 and 3, the interval of protrusions is a length g as shown in FIG. 3.

The height of the protrusions 4 is preferably set such that a neuron body 20a and a neural spine 20b can reach the lower portion of the protrusions 4, that is, the upper surface of the resin layer 3A. In the case of a general neuron 20, the height of the protrusions, if it is set at about 0.1 μm, is sufficient for the neuron to satisfy the condition. However, if the height is set at not less than 0.1 μm, the effect of the protrusions 4 can be more significantly exerted.

The lengthwise shape of the protrusions is not particularly limited and may be columnar, conical or inversed conical form. Furthermore, the shape of the outer circumference is not particularly limited and modification may be made.

The material for the protrusions is not particularly limited and should be selected depending upon a desired accuracy in processing, surface characteristics, optical characteristics, and strength. For example, the material for the protrusions may be appropriately selected from the resins, resin compositions, inorganic substances and biodegradable resins previously mentioned as examples of constitutional materials for the substrate base 2.

Note that the protrusions 4 and the resin layer 3 should be integrally formed as mentioned above; therefore, they are formed of the same material.

Various treatments may be applied to the surfaces of the protrusions 4 and resin layer 3 (that is, the surface of the neuron culture substrate 1) depending upon its purpose.

Examples of such treatments include, surface-coating with a biopolymer such as a protein, a metal thin film or the like for controlling the adhesion of the neurons 20 onto protrusions 4 and resin layer 3 and to protect the surface thereof; and at least one surface treatment selected from the group consisting of plasma treatment, UV irradiation, hydrophilic/hydrophobic treatment with a water-repellent and heating, addition of a predetermined functional group(s) such as hydroxyl group, amino group, sulfone group, thiol group, and carboxyl group, and rough-surface treatment by an oxidant. In particular, to accelerate adsorption of the neuron 20 onto the protrusions 4, coating of the-protrusions 4 with a protein such as polylysine, albumin, collagen, fibronectin, fibrinogen, vitronectin, or laminin is effective.

Such surface modification may be applied to a whole or part (limited area) of the surface of the protrusions 4 and resin layer 3. To be more specifically, a part of the protrusions 4 is modified in a different manner from that applied to the other part. Alternatively, the protrusions 4 and resin layer 3 are modified in different manners from each other. Furthermore, the top surfaces 4A of the protrusions 4 and the peripheral surfaces of the protrusions 4 may be modified in different manners.

In the embodiment, the protrusions 4 and resin layer 3 should be integrally formed. Furthermore, the substrate base 2 is also integrally formed with the protrusions 4 and resin layer 3. With the constitution, the strength of the neuron culture substrate 1 can be enhanced. On the other hand, when the substrate base 2 is desired to have different characteristics from the protrusions 4 and resin layer 3, they may be formed of mutually different materials.

The protrusions 4 are not necessarily formed only one surface of the substrate base 2 and may be formed on both surfaces of the substrate base 2 depending upon the culture method for neurons 20. Alternatively, when the substrate base 2 is constructed three dimensionally, protrusions 4 may be formed on each of the surfaces.

<A Method for Manufacturing Neuron Culture Substrate 1>

Referring now to the accompanying drawings, a method for manufacturing the neuron culture substrate 1 will be explained.

FIG. 4 is an illustration for explaining the steps of manufacturing the neuron culture substrate 1 in accordance with one of the methods, nano-imprinting.

As shown in FIG. 4(A), the resin layer 3 is formed on the substrate base 2. In this case, when the resin layer 3 is formed of an adhesive material, it can be adhered onto the substrate base 2 without applying any particular adhesive treatment. In the case of further increasing adhesiveness or the case where the resin layer 3 is not formed of an adhesive material, a predetermined treatment is applied to the upper surface of the substrate base 2 to enhance the adhesiveness with the resin layer 3. As an example of such treatment, use preferably made of a coating treatment with a functional group, such as silane coupling, plasma treatment, and coating treatment with a graft polymerized polymer and an adhesive polymer.

Subsequently, as shown in FIG. 4(B), the resin layer 3 arranged on the substrate base 2 is softened and then a mold 5 having a concave pattern 6 formed therein is impressed on the softened resin layer 3, thereby transferring the concave pattern 6 to the resin layer 3.

Thereafter, as shown in FIG. 4(C), the mold 5 is removed. In this manner, the neuron culture substrate 1 in which the protrusions 4 and resin layer 3 are integrally formed can be obtained.

The material for the mold 5 is appropriately selected from metals, inorganic substances such as carbon and silicon, and resin compositions, depending upon the materials of substrate base 2, protrusions 5, and accuracy in processing.

A method of forming the protrusion pattern 6 in the surface of the mold 5 is appropriately selected from the group consisting of cutting, nano-processing such as optical lithography, electron beam direct drawing, particle beam processing, and scanning probe processing, self-organization of fine particles, nano-imprinting from a master formed by these methods, casting, mold-processing represented by injection-molding, and plating.

The method for manufacturing the neuron culture substrate 1 is not limited to a nano-imprinting and may appropriately selected from the group of processing methods including cutting, printing, ion beam processing, electron beam processing, laser processing, optical lithography, casting, and injection molding, depending upon the materials for substrate base 2 and resin layer 3, and the accuracy of processing. Furthermore, when casting or injection molding is employed, the mold 5 formed by a method as mentioned above may be used.

Note that, the resin layer 3 is not necessarily formed on the neuron culture substrate 1 depending upon the manufacturing method. In this case, the protrusions 4 may be formed immediately on the upper surface 2A of the substrate base.

To the surface of the protrusions 4 and resin layer 3 thus formed, if necessary, surface modification may be applied. Examples of such surface modification include soaking, spin coating, vapor deposition, plasma polymerization, inkjet, and screen printing (modification of adding a new layer) and heating, light irradiation, electron irradiation, plasma treatment, and soaking treatment.

Such surface modification treatment is not necessarily performed after formation of the protrusions 4. For example, before the formation of protrusions 4, if the surface treatment is previously applied to the surface of the resin layer 3 or the protrusion pattern 6 of the mold, a modification treatment can be applied to the surfaces of the protrusions 4 and the resin layer 3 simultaneously with the formation of the protrusions 4.

In the foregoing, the neuron culture substrate 1 to be used in a method for culturing neurons according to the present invention has been explained. In the embodiment, growth of neurons can be controlled variously by varying an equivalent diameter r and the interval g of the protrusions 4 to be formed on the neuron culture substrate 1, thereby obtaining various morphologies.

Now, three specific embodiments will be explained below with reference to the drawings. In the embodiments, growth of neurons is controlled in three ways by using three types of neuron culture substrates 1 different in equivalent diameter r and interval g of the protrusions 4.

### Embodiment 1

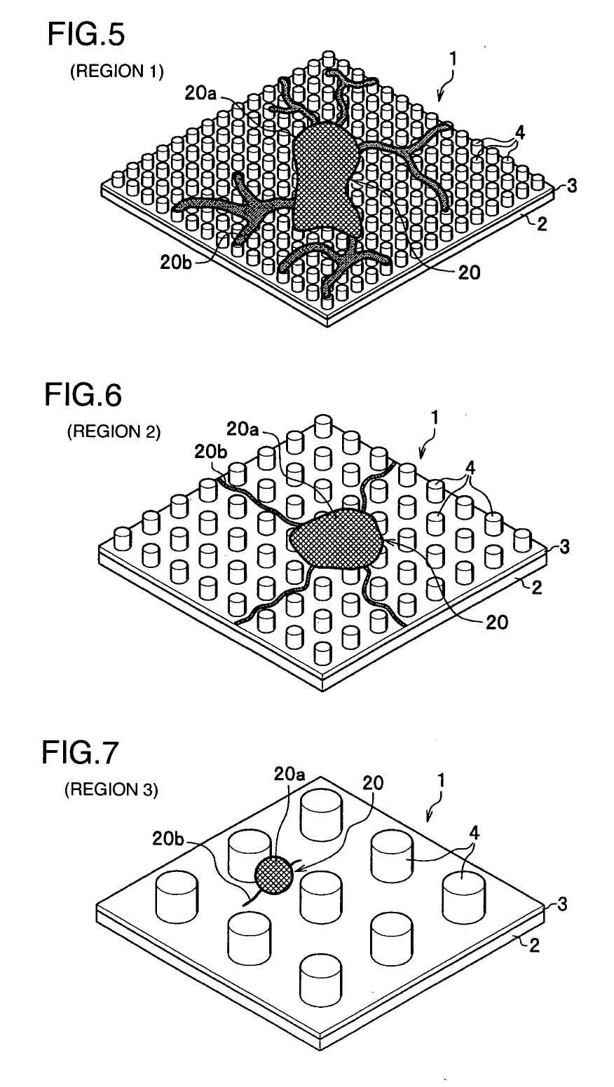

FIG. 5 is an illustration for explaining a method for culturing neurons according to Embodiment 1.

Note that the culture vessel 7 and medium 8 are omitted for brevity's sake in FIG. 5.

As shown in FIG. 5, in the neuron culture substrate 1 used in Embodiment 1, the equivalent diameter r of the protrusions 4 and the interval g of the protrusions 4 formed on the surface are set to be smaller than the diameter of the neuron 20 (cell body 20a) and the diameter of neurites 20b extending from the neuron 20.

When the neurons 20 are cultured by using the neuron culture substrate 1 thus constituted, the cell body 20a of the neuron 20 grows flatter and larger than the case of a flat substrate. The neurites 20b each extending from the neuron 20 grows on and along alignment of the protrusions. As a result, the neurites 20b are increased in diameter and branched in many directions.

The region constituted of predetermined protrusions 4 in the surface of the neuron culture substrate 1 and specified in Embodiment 1, is designated as Region 1. More specifically, when the neurons 20 are cultured within Region 1 shown in FIG. 5, adhesion between the neurons and the protrusions 4 increases. As a result, the neurons 20 can be cultured while controlling morphological growth, for example, increasing the thickness of neurites 20b and the number of branches.

### Embodiment 2

FIG. 6 is an illustration explaining a method for culturing neurons according to Embodiment 2.

Note that a culture vessel 7 and medium 8 are omitted for brevity's sake in FIG. 6.

As shown in FIG. 6, in the neuron culture substrate 1 used in Embodiment 2, the equivalent diameter r of the protrusions 4 and the interval g of the protrusions 4 formed on the surface are set to be smaller than the diameter of neuron 20 (cell body 20a) and the interval g of the protrusions is set to be larger than the diameter of neurites 20b extending from the neuron body 20a.

When the neurons 20 are cultured by using the neuron culture substrate 1 thus constituted, the neurites 20b extending from the neuron 20 grow through the interval of the protrusions along the alignment of protrusions 4. As a result, the neurites become longer than those cultured on a flat substrate.

The region constituted of predetermined protrusions 4 in the surface of the neuron culture substrate 1 and specified in Embodiment 2 is designated as Region 2. More specifically, when the neurons 20 are cultured within Region 2 shown in FIG. 6, the neurons 20 can be cultured while reducing the neurons 20 in size and controlling the direction of neurites extending from a neuron 20.

### Embodiment 3

FIG. 7 is an illustration explaining a method for culturing neurons according to Embodiment 3.

Note that a culture vessel 7 and medium 8 are omitted for brevity's sake in FIG. 7.

As shown in FIG. 7, in the neuron culture substrate 1 used in Embodiment 3, the equivalent diameter r of the protrusions 4 formed on the surface is set to be smaller than the diameter of neuron 20 and the interval g of the protrusions 4 is set at 0.4 to 2 times, more preferably 0.6 to 2 times as large as the diameter of neuron 20.

When the neurons 20 are cultured by use of the neuron culture substrate 1 thus constituted, the neurites 20b are prevented from extending from the neuron body 20a and the neuron 9 shrinks.

The region constituted of predetermined protrusions 4 in the surface of the neuron culture substrate 1 and specified in Embodiment 3 is designated as Region 3. More specifically, when the neurons 20 are cultured within Region 3 shown in FIG. 7, the neurons 20 can be cultured while suppressing the growth.

More specifically, when the neuron culture substrate 1 (see FIG. 2) having the region 3 shown in FIG. 7 is used, the growth of neurons can be suppressed (or terminated).

According to the Embodiments, the morphological growth of neurons can be controlled by defining the shape and interval of the protrusions on the culture substrate.

In conventional methods, a specific reagent such as a cytokine must be added to change the morphological growth of the neurons as mentioned above; however, such a reagent is not required in the Embodiments of the present invention. Therefore, side effect of a reagent is not necessary to take into consideration. Furthermore, when the embodiment is applied, the morphological growth of neurons can be locally controlled on the neuron culture substrate. As a result, the complicated and higher control of the morphological growth of the neurons can be attained.

In the foregoing, the present invent has been explained with reference to Embodiments; however, the present invention is not limited to the Embodiments and widely applied to various fields.

More specifically, the methods for culturing neurons shown in Embodiments 1 to 3 mentioned above can be performed in the same culture conditions, even though a neuron culture substrate 1 differs in constitution. Culturing can be performed simultaneously by placing several types of neuron culture substrates 1 different in constitution in a single culture vessel 7. In other words, there is provided a method for culturing neurons 20 by using a neuron culture substrate 1 having not less than two control regions, and also provided the neuron culture substrate 1. This is another embodiment different from Embodiments mentioned above.

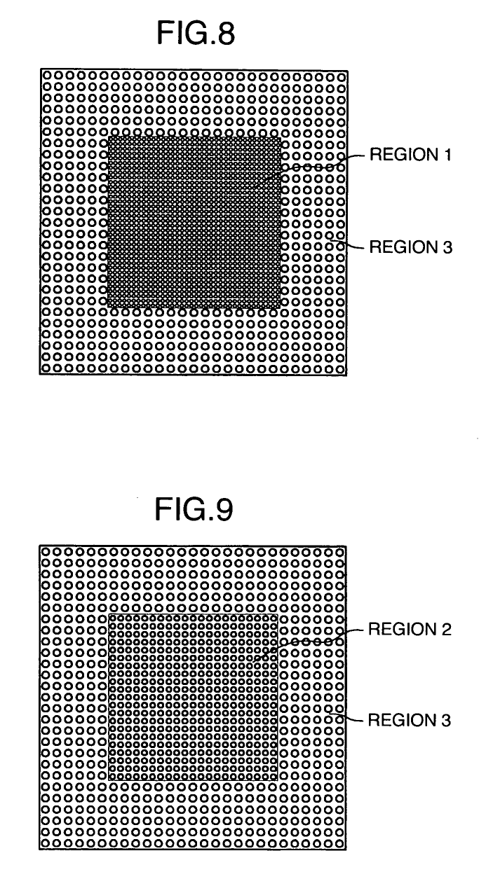

FIGS. 8 to 10 are top views of neuron culture substrates 1 in which regions 1 to 3 and an optional region 4 are formed in combination.

To explain more specifically, as shown in FIG. 8, a neuron culture substrate 1 can be formed so as to have Region 1 surrounded by Region 3. According to a culture method of neurons 20 by using the neuron culture substrate 1 shown in FIG. 8, neurons 20 having a large neuron body 20a with a thick neurite 20b can be obtained only within the center portion of the neuron culture substrate 1 after culturing.

Furthermore, as shown in FIG. 9, a neuron culture substrate 1 can be formed so as to have Region 2 surrounded by Region 3. According to a culture method of neurons 20 by using the neuron culture substrate 1 shown in FIG. 9, the lattice-form network of neurons can be formed only within the center portion of the neuron culture substrate 1.

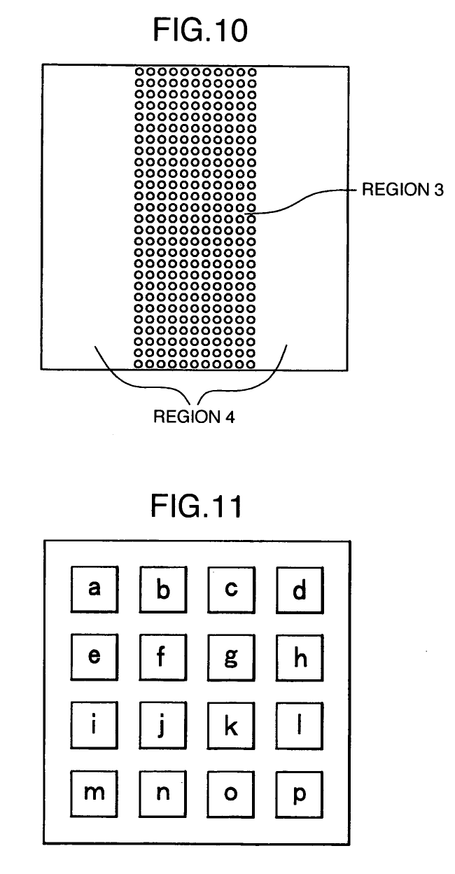

Moreover, as shown in FIG. 10, a neuron culture substrate 1 formed of two regions partitioned by Region 3 can be obtained. In FIG. 10, the optional Region 4 may be either Region 1 or 2, or a flat region having no protrusion 4. Alternatively, the Region 4 may have a structure such as a groove(s) as described in National Publication of International Patent Application No. 10-500031. According to a method for culturing neurons 20 using a neuron culture substrate 1 shown in FIG. 10, since the Region 3 would not inhibit growth of neurites 20b extending from outside, neurons 20 of two isolated regions (Region 4, Region 4) can be connected to each other only by the neurites 20b extending from the individual neuron bodies 20a.

The arrangements of Regions 1, 2, and 3 on the neuron culture substrate 1 are not limited to the aforementioned examples. Combination of Regions 1, 2, and 3 can be appropriately selected depending upon the required nature for neurons 20. Furthermore, protrusions may be arranged at random without providing clear boundary between regions.

As described in the above, neurons 20 can be cultured while controlling morphological growth of the neurons 20 individually in each region by arranging a plurality of control regions (including a case where control regions 1 to 3 (appear once) and optional region 4 (appears not less than once)) on a single neuron culture substrate 1.

Furthermore, the present invention may include a structure having a precut portion in a cell-growth suppression region (e.g., Region 3) of neurons 20, a region outside the cell-growth suppression region as viewed from the position at which neurons are provided, or the back surface of the neuron culture substrate 1. By virtue of this structure, a desired portion of the neuron system, which is constituted of the neuron culture substrate 1 and the neurons 20 cultured while suppressing its growth, can be easily taken out.

Thus, the neuron system according to the present invention as mentioned above and a method for manufacturing the system fall within the scope of the present invention. In other words, a system in which neurons 20 can be cultured with a desired morphology can be provided. Such a system is suitably used as a neuron graft piece and a neuron network.

### EXAMPLES

The present invention will be described in more detail below by way of Examples, which will not construed as limiting the present invent.

### Example 1

In this Example, a neuron culture substrate 1 was manufactured.

FIG. 11 is a top view of the neuron culture substrate 1 manufactured in Example 1.

A substrate base 2 was formed of non-alkaline glass (OA-10, manufactured by Nippon Electric Glass Co., Ltd.) in a size of 25 mm square and 0.7 mm thick.

The resin layer 3 was formed of polystyrene (manufactured by Sigma-Aldrich Japan) having a molecular weight of 3,000 to 6,000,000.

A polystyrene film of 1 μm thick was formed by spin coating on the substrate base 2 and heated at 90° C. for 5 minutes to vaporize the solvent. The resin layer 3 of a polystyrene thin film on the substrate base 2 was heated at 150° C. to soften the resin layer 3. Then, a mold 5 of single crystalline silicon (a crystal orientation <100>) of 20 mm square and 0.7 mm thick, in which a protrusion pattern 6 corresponding to the concave pattern as shown in FIG. 11 was formed in the substrate, was impressed on the softened resin layer 3 at a pressure of 10 MPa for 180 seconds. In this press step, the concave portions 6 of the mold was filled with the soften resin. Thereafter, the construct was cooled to 70° C. and the mold 5 was removed. In this manner, the neuron culture substrate 1 was obtained having protrusions 4 (shown in FIG. 11) formed on the surface.

Subsequently, the neuron culture substrate 1 was soaked in ethanol to dry it, subjected to UV sterilization for 3 hours, soaked in polylysine solution (50 mg/0.1M boric acid solution, pH=8.3) for one hour and washed with pure water. In this manner, the protrusions 4 were coated with polylysine.

Note that the mold 5 used herein was formed of a single crystalline silicone (a crystal orientation <100>) of 20 mm square and 0.7 mm thick and the concave pattern of the mold 6 was formed by optical lithography.

As shown in FIG. 11, the neuron culture substrate 1 had 16 control regions having predetermined protrusions. The size of each region was 3 mm square. These protrusions 4 of each region were arranged in the form of a two dimensional tetragonal lattice.

The protrusions 4 all had a height of 1 μm. The diameter r of the protrusions varies within 0.25 μm to 25.0 μm. There are two types of regions: one is a region where protrusions 4 were arranged at the intervals g equal to the diameter r, and the other is a region where protrusions were arranged at intervals g twice as large as the diameter r.

The equivalent diameters r of the protrusions 4 and the intervals g of the protrusions 4 formed on the neuron culture substrate 1 in this Example are listed in Table 1.

TABLE 1

| | Region | | | | | | | |
|---|---|---|---|---|---|---|---|---|
| | a | b | c | d | e | f | g | h |
| Equivalent | 0.25 | 0.50 | 0.75 | 1.0 | 2.0 | 5.0 | 10 | 25 |
| diameter r of | | | | | | | | |
| protrusion (μm) | | | | | | | | |
| Interval g of | 0.25 | 0.50 | 0.75 | 1.0 | 2.0 | 5.0 | 10 | 25 |
| protrusions (μm) | | | | | | | | |
| | Region | | | | | | | |
| | i | j | k | l | m | n | o | p |
| Equivalent | 0.25 | 0.50 | 0.75 | 1.0 | 2.0 | 5.0 | 10 | 25 |
| diameter r of | | | | | | | | |
| protrusion (μm) | | | | | | | | |
| Interval g of | 0.50 | 1.0 | 1.5 | 2.0 | 4.0 | 10 | 20 | 50 |
| protrusions (μm) | | | | | | | | |

### Example 2

In this Example, neurons were cultured by using the neuron culture substrate 1 prepared in Example 1 and the morphological growth of neurons was evaluated.

First, a procedure for preparing neurons 20 used in this Example was shown below.

A fetus was taken out from a mouse of 14th day of pregnancy and then the brain was excised out from the fetus. Only the brain cortex was separated from the cerebral hemisphere and tissue pieces of the brain cortex were collected in a 15 ml tube containing a medium (Opti-MEM (manufactured by Invitrogen Corporation), and 2-mercaptoethanol (manufactured by Invitrogen Corporation)). The cells of the tissue pieces were dispersed while pipetting by a Pasteur pipette whose top was rounded by burner flame. Thereafter, the number of cells were counted by a hemocytometer and stained with trypan blue (manufactured by Invitrogen Corporation) to confirm that the cells had appropriate viability.

The neuron culture substrate 1 coated with polylysine obtained in Example 1 was placed in a vessel 7 such as a cell culture dish and the neurons 20 taken from the brain cortex tissue of 14th-day murine fetus were seeded with a density of 2.0×10⁴/cm². Culture at the first day was performed by using a serum medium (Opti-MEM, 10% FBS (manufactured by Invitrogen Corporation), 55 μM 2-mercaptoethanol) was used as the medium 8. After the 2nd day when the cells were fixed, culture was performed by using a non-serum medium (Opti-MEM, B27 supplement (Invitrogen Corporation), 55 mM 2-mercaptoethanol) was used. Culture was performed in a CO₂ incubator (CO₂ concentration: 5%, a temperature of 37° C., relative humidity: 80%). After culturing for 7 days, the morphology of the cultured neurons 20 was evaluated by an inverted microscope or a scanning microscope.

The regions a to p of this Example were evaluated for morphology of the neuron body 20a (described as neuron body shape in Table 2), the total number of neurites 20b per cell, the number of branched neurites 20b per cell, an average length of the neurites 20b, an average thickness of the neurites 20b, and degree of orientation along the extension direction of the neurites.

The term “the degree of orientation along the extension direction” refers to as the ratio of neurites extending straightforward to total neurites.

As a comparative example, the neurons 20 were seeded on a flat polystyrene substrate, cultured in the same conditions, and evaluated.

The evaluation results are shown in Table 2.

TABLE 2

| | | Structure of | | Morphologies of neuron body and neurites | | | | | |
|---|---|---|---|---|---|---|---|---|---|
| | | protrusion | | | Neurite (average per cell) | | | | |
| | | Equivalent | | | | The number | | | Degree of |
| | | diameter r | Interval | Shape of | Total | of neurite | Length | Thickness | orientation |
| Region | | (μm) | g (μm) | neuron body | number | branchings | (μm) | (μm) | (%) |
| Example | a | 0.25 | 0.25 | Flat shape | 3.4 | 8.0 | 220 | 1.5 | 0 |
| | b | 0.50 | 0.50 | Flat shape | 2.5 | 5.2 | 205 | 1.4 | 22 |
| | c | 0.75 | 0.75 | Flat shape | 2.6 | 1.8 | 255 | 1.0 | 56 |
| | d | 1.0 | 1.0 | Flat shape | 2.1 | 1.0 | 295 | 0.8 | 52 |
| | e | 2.0 | 2.0 | Flat shape | 2.0 | 2.0 | 110 | 0.6 | 56 |
| | f | 5.0 | 5.0 | Spherical form | 1.2 | 0.0 | 20 | 0.2 | 0 |
| | g | 10 | 10 | Spherical form | 1.6 | 0.0 | 15 | 0.3 | 0 |
| | h | 25 | 25 | Spindle form | 2.0 | 4.0 | 120 | 0.2 | 0 |
| | i | 0.25 | 0.50 | Flat shape | 2.9 | 6.3 | 235 | 1.5 | 0 |
| | j | 0.50 | 1.0 | Flat shape | 3.1 | 3.5 | 210 | 1.4 | 31 |
| | k | 0.75 | 1.5 | Flat shape | 2.2 | 2.0 | 280 | 0.9 | 50 |
| | l | 1.0 | 2.0 | Flat shape | 2.0 | 1.2 | 130 | 0.5 | 70 |
| | m | 2.0 | 4.0 | Spherical form | 2.0 | 0.0 | 25 | 0.2 | 0 |
| | n | 5.0 | 10 | Spherical form | 1.8 | 0.0 | 20 | 0.2 | 0 |
| | o | 10 | 20 | Spindle form | 2.0 | 4.0 | 20 | 0.3 | 0 |
| | p | 25 | 50 | Spindle form | 2.4 | 3.0 | 110 | 0.2 | 0 |

| | | | | | | | |
|---|---|---|---|---|---|---|---|
| Comparative Example | No protrusion (flat) | Spindle form | 4.6 | 3.2 | 130 | 0.2 | 0 |

As shown in Table 2, in the regions a, b and i, the shape of the cell bodies 20a was flat. The neurite 20b extended while propagating over the protrusions 4 and repeating diversion (branch). The neurite was thicker than that obtained in a flat-substrate culture. The diameter of neuron bodies 20a in general murine neurons fall in the range of 2 to 20 μm and the diameter of neurites 20b in the range of 0.3 to 2.0 μm. In the regions mentioned above, the diameter r of the protrusions 4 and the interval g of protrusions 4 are smaller than the diameters of the neuron bodies and spines 20b. As mentioned above, it was demonstrated that the morphological growth of the neurons 20 can be controlled by the presence of protrusions 4 thus constituted, as shown in Embodiment 1.

As shown in Table 2, in the regions c, d, e, k and 1, the shape of the cell bodies 20a was flat. However, the number of branches of the neurite 20b was smaller than that in a flat substrate culture. It was demonstrated that the neurite extended straightforward through the space between the protrusions along the alignment of the protrusions while suppressing diversion (branch). As described above, the diameter of the neuron bodies 20a in general murine neurons falls in the range of 2 to 20 μm and the diameter of neurites 20b in the range of 0.3 to 2.0 μm. Therefore, in the regions mentioned above, the diameter r of the protrusion 4 and the interval g of protrusions 4 are smaller than the diameter of the neurons and the interval g is larger than the diameter of the neurites 20b. As mentioned above, it was demonstrated that the morphological growth of the neurons 20 can be controlled by the presence of protrusions 4 thus constituted, as shown in Embodiment 2.

As shown in Table 2, in the regions f, g, m and n, cell bodies 20a shrunk and exhibited a spherical shape smaller than usual. The growth of the neurite 20b was suppressed. The number and length of branches are smaller than those in a flat substrate culture. As described above, the diameter of the neuron bodies 20a in general murine neurons falls in the range of 2 to 20 μm and the diameter of neurites 20b in the range of 0.3 to 2.0 μm. In the regions mentioned above, the diameter r of the protrusions 4 is smaller than the diameter of the neurons and the interval g is 0.4 to 2.0 times as large as the diameter of the neuron. As mentioned above, it was demonstrated that the morphological growth of the neurons 20 can be controlled by the presence of protrusions 4 thus constituted, as shown in Embodiment 3.

As shown in Table 2, in the regions h, l and o, no significant difference in shape between the neurons 20 and the neurons cultured on a flat substrate. This means that since the diameter r and the interval g of the protrusions are sufficiently larger than the diameter of the neurons in these regions, cells were cultured in the same culture conditions as on a flat substrate.

According to Examples, it was demonstrated that neurons 20 can be cultured while controlling the morphological growth thereof by specifying the shape of protrusions 4 formed on the neuron culture substrate 1 even if they are cultured in the same conditions including temperature, culture time, and medium. More specifically, according to the Examples, various patterns of neuron network can be formed by using a neuron culture substrate 1 that is constructed by arranging protrusions having different characteristics on the substrate in a desired pattern.

It should be further understood by those skilled in the art that although the foregoing description has been made on embodiments of the invention, the invention is not limited thereto and various changes and modifications may be made without departing from the spirit of the invention and the scope of the appended claims.

1. A method for culturing neurons comprising providing a culture medium and neurons on a neuron culture substrate made of an organic polymer and culturing the neurons in corresponding culture conditions, wherein a culture surface of the neuron culture substrate has a plurality of protrusions, and a shape, an interval or both of the plurality of protrusions are controlled to control morphological growth of the neurons.

2. The method for culturing neurons according to claim 1, wherein the morphological growth of the neurons to be controlled is at least one of adhesion between the neurons and the neuron culture substrate, a shape of neuron bodies, a thickness, a length, a number of branches, and an extension direction of neurites extending from the neuron bodies, and growth suppression of the neurons.

3. The method for culturing neurons according to claim 1, wherein an equivalent diameter of the plurality of protrusions is controlled to be smaller than a diameter of the neurons to control adhesion between the neurons and the neuron culture substrate and a shape of neuron bodies.

4. The method for culturing neurons according to claim 1, wherein an equivalent diameter of and the interval between the plurality of protrusions are controlled to be smaller than a diameter of the neurons to be cultured and a diameter of neurites extending from the neurons to increase a thickness and number of branches of the neurites.

5. The method for culturing neurons according to claim 1, wherein an equivalent diameter of and the interval between the protrusions are controlled to be smaller than a diameter of the neurons to be cultured, and the interval between the protrusions is controlled to be larger than a diameter of neurites extending from the neurons to control an extension direction of the neurites extending from the neurons.

6. The method for culturing neurons according to claim 1, wherein an equivalent diameter of the plurality of protrusions is controlled to be smaller than a diameter of the neurons, and an interval between the protrusions is controlled to be 0.4 to 2 times the diameter of the neurons to suppress the growth of the neurons.

7. A neuron culture substrate for use in culturing neurons, wherein the neuron culture substrate is made of an organic polymer and has a culture control region for controlling morphological growth of neurons formed by a plurality of protrusions in a surface of the neuron culture substrate on which the neurons are provided, and the culture control region is at least one region selected from the group consisting of:
(a) at least one region formed by a plurality of protrusions, in which an equivalent diameter of and an interval between the protrusions are smaller than a diameter of the neurons to be cultured and a diameter of neurites extending from the neurons;
(b) at least one region formed by a plurality of protrusions, in which an equivalent diameter of and an interval between the protrusions are smaller than a diameter of the neurons to be cultured, and the interval between the protrusions is larger than a diameter of neurites extending from the neurons; and
(c) at least one region formed by a plurality of protrusions, in which an equivalent diameter of the protrusions is smaller than a diameter of the neurons, and an interval between the protrusions is 0.4 to 2 times the diameter of the neurons.

8. The neuron culture substrate according to claim 7, wherein the neuron culture substrate comprises a culture region (d) in which a plurality of the protrusions are not formed in a surface of the neuron culture substrate on which the neurons are cultured.

9. The neuron culture substrate according to claim 7, comprising
at least one first region formed by a plurality of protrusions in which the equivalent diameter of and the interval between the protrusions are smaller than the-diameter of the neurons to be cultured and the diameter of the neurites extending from the neurons; and
at least one second region formed by a plurality of protrusions in which the equivalent diameter of the protrusions is smaller than the diameter of the neurons, and the interval between the protrusions is 0.4 to 2 times the diameter of the neurons.

10. The neuron culture substrate according to claim 7, comprising at least one region selected from the group consisting of
at least one first region formed by a plurality of protrusions in which the equivalent diameter of and the interval between the protrusions are smaller than the diameter of the neurons to be cultured, and the interval between the neurites is larger than the diameter of neurites extending from the neurons; and
at least one second region formed by a plurality of protrusions in which the equivalent diameter of the protrusions is smaller than the diameter of the neurons, and the interval between the protrusions is 0.4 to 2 times the diameter of the neurons.

11. The neuron culture substrate according to claim 7, wherein the culture control region or the culture region comprises not less than one region partitioned by at least one region formed by the plurality of protrusions in which the equivalent diameter of the protrusions is smaller than the diameter of the neurons, and the interval between the protrusions is 0.4 to 2 times the diameter of the neurons.

12. The neuron culture substrate according to claim 7, wherein the surface of the neuron culture substrate on which the neurons are provided is surface-treated for accelerating adhesion of the neurons.

13. The neuron culture substrate according to claim 7, wherein a precut portion is provided in a back surface of the neuron culture substrate or the culture control region of the neurol culture substrate formed by the plurality of protrusions in which the equivalent diameter of the protrusions is smaller than the diameter of the neurons and the interval between the protrusions of 0.4 to 2 times the diameter of the neurons, or a region outside the region as viewed from the side on which the neurons are provided.

14. The method for culturing the neurons according to claim 1, comprising using the neuron culture substrate according to claim 7.

15. Neurons cultured on a neuron culture substrate made of an organic polymer, wherein the neuron culture substrate has a plurality of protrusions, and a shape of and an interval between the neurons are controlled to control at least one of shape of cell bodies, thickness, length, the number of branches, and extension direction of neurites.

16. The neurons according to claim 15, wherein the diameter of at least a part of the protrusions is smaller than the diameter of the neurons.

17. The neurons according to claim 15, wherein, the equivalent diameter of and the interval between at least a part of the protrusions are smaller than the diameter of the neurons and the diameter of the neurites extending from the neurons.

18. The neurons according to claim 15, wherein the equivalent diameter of and the interval between at least a part of the protrusions are smaller than the diameter of the neurons, and the interval is larger than the diameter of the neurites extending from the neurons.

19. The neurons according to claim 15, wherein the equivalent diameter of at least a part of the protrusions is smaller than the diameter of the neurons, and the interval is 0.4 to 2 times the diameter of the neurons.

20. A neuron system composed of a neuron culture substrate and a neuron network formed on the neuron culture substrate, wherein a culture surface of the neuron culture substrate has a plurality of protrusions, and morphological growth of the neurons on the neuron culture substrate is controlled by specifying a shape, an interval or both of the protrusions.

21. A method for manufacturing a neuron system composed of a neuron culture substrate and a neuron network formed on the neuron culture substrate, comprising:
fixing neurons onto a culture surface of the neuron culture substrate having a plurality of proteusions whose shape, interval or both is specified; and
culturing the fixed neurons in culture conditions corresponding to the neurons to form the neurons on the neuron culture substrate, while controlling morphologial growth.
